(19) United States
(12) Patent Application Publication
(10) Pub. No.: US 2004/0138237 A1
(43) Pub. Date: Jul. 15, 2004

(54) Novel injectable depot formulations

(75) Inventor: Jaymin C. Shah, Waterford, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/690,078

(22) Filed: Oct. 21, 2003

Related U.S. Application Data
(60) Provisional application No. 60/421,473, filed on Oct. 25, 2002.

(51) Int. Cl. A61K 31/519; A61K 9/14

(52) U.S. Cl. 514/259.41; 424/486

(57) ABSTRACT

An injectable depot formulation that is viscous, or becomes viscous in situ, comprising a solubilized aryl-heterocyclic pharmaceutical compound, such as ziprasidone, is provided.

This application claims priority under 35 USC 119(e) of U.S. Provisional 60/421,473, filed Oct. 25, 2002.

### FIELD OF INVENTION

The invention pertains to injectable depot formulations for aryl-heterocyclic compounds, such as arylpiperazinyl-C₂ and -C₄ alkyleneheterocycle compounds, including ziprasidone; and methods for making same. The injectable depot formulations of the invention permit controlled release of the active aryl-heterocyclic substances over prolonged periods of time after administration to a patient via intramuscular (IM) injection, for example.

### BACKGROUND OF THE INVENTION

Certain aryl-heterocyclic compounds are known to have psychotropic effects. Ziprasidone in particular is a chlorooxyindole class aryl-heterocyclic that is an atypical anti-psychotic agent often prescribed for the treatment of schizophrenia. Atypical anti-psychotics such as ziprasidone offer distinct advantages over traditional anti-psychotic medications insofar as they are associated with lower incidences of side effects, such as extrapyramidal symptoms (EPS), and confer greater efficacy of treatment to patients who are otherwise not responsive to more traditional drug therapies. Certain illnesses, such as schizophrenia, can be particularly difficult to medicate inasmuch as they are considered to be heterogeneous diseases whereby not all patients react similarly to the same treatment regimen. Exacerbating this is the problem that commonly attends long term treatment of schizophrenia; namely, non-compliance by patients with their dosage schedules. Indeed, it is conventionally thought that substantial numbers of schizophrenic patients are not or only partially compliant with their medication. Poor compliance can cause relapse into the psychotic condition thereby negating whatever benefits were achieved through treatment in the first place.

Where patient compliance is an issue, resort is sometimes had to long acting dosage forms of the medication. That is, dosage forms where a single administration leads to a sustained release of the medication over an extended period of time. This, in turn, simplifies the dosage regimen that a patient need adhere to, thus reducing the opportunity for non-compliance as occurs with a more rigorous schedule. Among such dosage forms is the depot formulation, which can be administered in various ways including intramuscularly by injection. The depot dosage injection is specifically formulated to provide slow absorption of the drug from the site of administration, often keeping therapeutic levels of same in the patients system for days or weeks at a time. But there are instances where the use of a depot form has not been available. For example, in current practice, ziprasidone is administered once or twice daily in the form of an immediate release (IR) capsule for acute and long term treatment of schizophrenia; or is administered in intramuscular immediate release injection form for acute control of agitation in schizophrenic patients. Ziprasidone is poorly soluble. Indeed, for the intramuscular immediate release formulation aforesaid, even ziprasidone mesylate, which is generally soluble relative to other known ziprasidone salts, has to be solubilized further, presently with the use of cyclodextrins as described in U.S. Pat. No. 6,232,304 incorporated herein by reference, to render it efficacious.

In the case of ziprasidone, it has been found that its poor solubility, which suggests amenability to a depot formulation where the drug should not be too soluble (to avoid burst) and release must be prolonged, does not in fact provide adequate pharmacokinetic exposure when constituted as such in a depot formulation.

Consequently, there is a need for a depot formulation for aryl-heterocyclic compounds, such as ziprasidone, which can provide drug delivery over a sustained period of time at concentrations efficacious for treatment of, e.g. schizophrenia, in mammals including humans.

### SUMMARY OF THE INVENTION

The invention is premised on the finding that the solubilized forms of aryl-heterocyclics typically associated with (or with solubilized levels of active ingredient even greater than) immediate release, can be surprisingly fabricated into depot formulations. Hence in one aspect, the present invention is directed to an injectable depot formulation comprising a solubilized aryl-heterocyclic compound, such as ziprasidone, and a viscosity agent.

### DETAILED DESCRIPTION OF THE INVENTION

The injectable depot formulation of the invention provides significantly higher solubility of the aryl-heterocyclic drug in the formulation. The invention achieves this improved drug loading and delivery by using solubilizers cooperatively with viscosity agents to obtain the controlled release typifying a depot effect.

The invention is useful in treating psychotic illnesses such as schizophrenia in mammals, including humans in need of such treatment. The invention is also useful in treating other disorders and conditions, the treatment of which is facilitated by ziprasidone administration. Thus the present invention has application where ziprasidone use is indicated as e.g. in U.S. Pat. Nos. 6,245,766; 6,245,765; 6,387,904; 5,312,925; 4,831,031; and European EP 0901789 published Mar. 17, 1999, all of which are incorporated herein by reference.

The drug compounds contemplated for use in the present invention are aryl-heterocyclics, preferably those that have pharmacologic activity, e.g. psychotropic effects. Without limitation, an embodiment of an aryl-heterocyclic compound subject to the practice of the present invention has the structure:

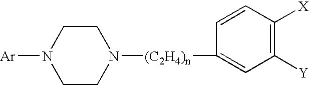

wherein

Ar is benzoisothiazolyl or an oxide or dioxide thereof, each optionally substituted by one fluoro, chloro, trifluoromethyl, methoxy, cyano, or nitro: n is 1 or 2; and

X and Y together with the phenyl to which they are attached form benzothiazolyl; 2-aminobenzothiazolyl; benzoisothiazolyl; indazolyl; 3-hydroxyindazolyl; indolyl; oxindolyl optionally substituted by one to three of (C₁-C₃)alkyl, or one of chloro, fluoro or phenyl, said phenyl optionally substituted by one chloro or fluoro; benzoxazolyl; 2-aminobenzoxazolyl; benzoxazolonyl; 2-aminobenxozazolinyl; benzothiazolonyl; benzoimidazolonyl; or benzotriazolyl. Representative examples of compounds falling within the foregoing definition are found in U.S. Pat. No. 4,831,031 incorporated herein by reference.

In one practice, the invention preferably applies to the above compounds wherein X and Y together with the phenyl to which they are attached form oxindole; more preferably, the oxindole moiety is 6-chlorooxindole-5-yl. In another preferred practice, Ar is benzoisothiazoyl; in still another preferred practice, n is 1. A particularly preferred aryl-heterocyclic to which the invention pertains is ziprasidone, which has the structure:

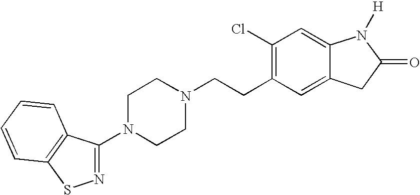

Although the aryl heterocyclic compound described herein may be constituted as a free base, it is preferred if aryl-heterocyclic compound is present as a pharmaceutically acceptable salt. The term “salt” in this regard intends pharmaceutically acceptable acid addition salts of aryl-heterocyclics, including ziprasidone. For purposes of preparing the formulation of the invention, the salts can be anhydrous or in the form of one or more solvates, such as hydrates, including mixtures thereof. The salts may also occur in different polymorphic forms. By way of exemplification only, mesylate salts of the aryl heterocyclic ziprasidone may be present in dihydrate or trihydrate forms as disclosed in U.S. Pat. Nos. 6,110,918 and 6,245,765 both of which are incorporated herein by reference. Without limitation, preferred salts are selected from the group consisting of the tosylate, tartrate, napsylate, besylate, aspartate, esylate and mesylate salt. In an especially preferred practice, the aryl heterocyclic is ziprasidone mesylate, more preferably in the trihydrate form.

The term “ziprasidone”, as used herein, unless otherwise indicated, includes ziprasidone free base and all pharmaceutically acceptable salts of ziprasidone, including all polymorphic forms thereof.

The injectable depot formulation of the present invention provides delivery of the aryl heterocyclic active agent at concentrations effective for treatment of illnesses such as schizophrenia over a sustained period of time, i.e. for a period of time beyond that which is obtained by immediate release injection systems. Thus by way of further definition the injectable depot formulation of the present invention provides for example efficacious plasma levels of active agent for at least about 8 hours using typical injection volumes, e.g. about 0.1 ml to about 3 ml, about 1 ml to about 2 ml being usual. Preferably, the sustained period provided by the invention is at least about 24 hours; more preferably up to about 1 week; still more preferably from about 1 week to about 2 weeks or more including up to about 8 weeks using the injection volumes aforesaid.

For example, in the case of ziprasidone, the practice of the invention can deliver at least 0.5 to about 350 mgA per ml injection. Typically, the injection volume is from about 1 ml to about 2 ml, thereby providing delivery of from about 0.5 mgA to about 700 mgA ziprasidone over a sustained period of time. More preferably, about 10 mgA to about 560 mgA ziprasidone is provided per injection over a sustained period of time, even more preferably about 280 mgA to about 560 mgA. As above described, an injection of the subject depot formulation can result in a sustained delivery of such amounts of ziprasidone over a period of time. In one embodiment, the period of time is at least about 8 hours, more preferably at least about 24 hours, even more preferably at least about 1 week. In another embodiment, the injection provides a sustained delivery of such amounts of ziprasidone for a period of time of at least about 2 weeks. In a further embodiment, the injection provides a sustained delivery of such amounts of ziprasidone for a period of time of up to about 8 weeks.

In the practice of the invention the aryl heterocyclic compound is solubilized. The term “solubilized” and related variations of same as used herein means that the heterocyclic has a solubility in water that is in excess of its free or salt forms to a degree sufficient to provide the prolonged (depot) duration of systemic exposure of active agent at the therapeutic levels contemplated by the invention. Without limitation, the heterocyclic can be “solubilized” using a cyclodextrin or other solubilizer to achieve the increased solubility contemplated herein. Thus the heterocyclic may be partly or fully solubilized. For convenience, the invention will now be further described exemplifying ziprasidone as the aryl heterocyclic compound. It is to be understood that the following discussion does not limit the scope of the invention and that the techniques hereinafter described appertain to and can be adapted for the family of aryl heterocyclics as disclosed herein. Other techniques that achieve the purposes stated can also be implemented and are envisioned as within the inventive practice.

The term “mgA/ml” as used herein relates to the weight (in mg) of aryl-heterocyclic compound, e.g. ziprasidone, per ml of composition to which the term is being applied. For ziprasidone free base, molecular weight=412.9.

In one embodiment, ziprasidone concentration is at least about 0.5 to about 350 mgA/ml, for example about 60mgA/ml, in the depot formulation of the present invention which can include amounts in solution and amounts in suspension as appertain. More preferably for ziprasidone, concentration is between about 70 mgA/ml to about 280 mgA/ml depot formulation, including between about 140 mgA/ml and about 210 mgA/ml, of depot formulation; higher concentrations are also within the scope of the invention. Various techniques to solubilize ziprasidone to obtain these levels of concentration involve, without limitation, the use of cyclodextrins and other solubilizers.

The preferred solubilizer (to form the solubilized aryl-heterocyclic compound of the invention) is a cyclodextrin. Cyclodextrins are cyclic oligosaccharides with hydroxyl groups on the outer surface and a void cavity in the center. The outer surface is usually hydrophilic hence cyclodextrins are soluble in water. The void on the other hand is typically hydrophobic. Cyclodextrins have the ability to form complexes with guest molecules, such as ziprasidone. Cyclodextrins contemplated by the invention include without limitation: α, β,γ-cyclodextrins, methylated cyclodextrins, hydroxypropyl-β-cyclodextrin (HPBCD), hydroxyethyl-β-cyclodextrin (HEBCD), branched cyclodextrins in which one or two glucoses or maltoses are enzymatically attached to the cyclodextrin ring, ethyl- and ethyl-carboxymethyl cyclodextrins, dihydropropyl cyclodextrins, and sulfoalkyl ether cylcodextrins, such as sulfobutyl ether-β-cyclodextrin (SBECD). The cyclodextrins can be unsubstituted or substituted in whole or in part as known in the art; mixtures of cyclodextrins are also useable. The preferred cyclodextrins for the depot formulation of the invention include γ-cyclodextrin, HPBCD, SBECD or mixtures thereof; SBECD being most preferred.

Cyclodextrin complexes with ziprasidone can be rendered soluble in water as described in U.S. Pat. No. 6,232,304 incorporated by reference above. For purposes of the invention, a pre-formed (solid) complex of cyclodextrin and ziprasidone can be employed, or the cyclodextrin can be presented separately into the depot formulation to solubilize the ziprasidone, such as by adding the cyclodextrin conjointly or in admixture with the viscosity agent or other components.

Viscosity agents include those known in the art such as viscosified water, pharmaceutically acceptable oils and oil-based agents, polymeric agents and other non-aqueous viscous vehicles. Preferred viscosity agents include without limitation: cellulose derivatives, polyvinylpyrrolidone, alginates, chitosan, dextrans, gelatin, polyethylene glycols, polyoxyethylene ethers, polyoxypropylene ethers, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers and terpolymers of the foregoing, and mixtures thereof. Preferred cellulose derivatives include methyl cellulose, sodium carboxymethyl cellulose (NaCMC) and hydroxypropyl methyl cellulose. Preferred polylactides, polyglycolides, copolymers and terploymers thereof include poly-lactic-co-glycolic acid (PLGA). Also contemplated as viscosity agents for the present invention are in situ gelling systems, e.g. stearic acid (SA) and N-methyl pyrrolidone (NMP) combinations, sucrose acetate isobutyrate, PLGA.

In the practice of the invention, the viscosity agent is present in an amount effective to provide the depot effect contemplated herein. Among other considerations in this regard, an effective amount of viscosity agent is that amount necessary to provide the depot formulation of the invention with a viscosity of greater than about 3.2 centipoise (cps); more preferably between about 20 and about 200 cps; still more preferably between about 30 and about 165 cps.

In a first embodiment of the invention, ziprasidone is solubilized with a cyclodextrin such as SBECD, wherein the cyclodextrin is present in a concentration of up to about 60% w/v, more preferably, a concentration of about 40% w/v, still more preferably a concentration of about 30%. In another embodiment, the depot formulation comprises a concentration of cyclodextrin, e.g. SBECD, of from about 5% to about 35%, especially from about 10% to about 20%. In a preferred aspect, the depot formulation comprising a cyclodextrin in this regard takes the form of an aqueous suspension, wherein the viscosity agent, e.g. NaCMC or the like, is present in water, e.g. sterilized water for injection, in an amount sufficient to render the viscosity of the depot formulation greater than 3.2 cps, preferably between about 20 cps to about 200 cps, more preferably, between about 30 cps to about 165 cps. For example, NaCMC can be present in an amount of about 0.1% w/v to about 3% w/v, preferably from about 0.5% w/v to about 2%. Optionally, the aqueous suspension depot formulation further comprises a pharmaceutically acceptable surfactant, for example a polyoxyethylene sorbitan ester such as Polysorbate 80 (Tween 80). The pharmaceutically acceptable surfactant can be present in an amount e.g. of up to about 1% w/v; preferably about 0.01 to about 0.1%.

In one practice of this first embodiment, the depot formulation can be in kit form as described in commonly-owned U.S. Provisional Application 60/421,295 filed Oct. 25, 2002, and as described in patent applications claiming priority of U.S. 60/421,295, the entire contents of which are incorporated herein by reference. By way of example only, the kit includes a first component of e.g. dry ziprasidone mesylate trihydrate in an amount sufficient to provide a dosage within the ranges described above, i.e. from about 0.5 mgA to about 350 mgA per ml depot formulation; and separately a second component comprised of viscous aqueous vehicle, such as NaCMC and a sufficient amount of water to render a total volume for injection of about 1 to about 3 ml, preferably 1 to about 2 ml; and SBECD or another cyclodextrin in an amount of about 5 to about 35% w/v to solubilize the ziprasidone. Optionally, a pharmaceutically acceptable surfactant, such as, without limitation, a polyoxyethylene sorbitan ester such as Polysorbate 80 (Tween 80) can be included with the viscosified NaCMC-water to improve wetting of the dry ziprasidone when the contents of the two elements are admixed together to form the injectable depot formulation of the invention. A depot formulation formulated as such can, in one embodiment, deliver at least about 10 to about 30 mg per day of ziprasidone for at least about 8 hours, preferably at least about 24 hours, more preferably at least about 1 week, even more preferably at least about 2 weeks.

In a second embodiment of the invention, a high concentration of cyclodextrin, e.g. SBECD, is utilized. In this embodiment, the cyclodextrin serves as both solubilizer and viscosity agent. That is, at high concentrations of cyclodextrin, the complex with ziprasidone forms an aqueous solution having a viscosity sufficiently high to provide a depot formulation. Ziprasidone in this regard is solubilized with a cyclodextrin concentration of greater than about 50% w/v, preferably from about 50% w/v to about 60% w/v; more preferably, the cyclodexdrin concentration is between about 55% to about 60% w/v, e.g. about 56% to about 57% w/v. Thus in one practice of this embodiment, about 80 mgA/ml of ziprasidone is solubilized with about 56% SBECD to create an aqueous solution suitable for an injectable depot formulation with a viscosity of about 22.6 cps and higher. In an optional practice, a crystallization inhibitor such as polyvinyl pyrrolidone (e.g. PVP 30) and the like may be added to delay crystallization and enhance the physical stability of the depot formulation.

In a third embodiment of the invention, a complex of ziprasidone and a cyclodextrin is formed and isolated as a solid. This solubilized solid complex can then be suspended in a suitable viscosity vehicle, including non-aqueous viscous agents in which the ziprasidone-cyclodextrin complex is not soluble. Without limitation, a solid preformed complex can be obtained by lyophilizing the high concentration solution of the second embodiment described above. The lyophilized complex can be suspended in non-aqueous viscosity agents including without limitation: sesame seed oil, including aluminum monostearate (ALMS) gelled sesame seed oil; and in situ gelling systems such as e.g. stearic acid (SA) and NMP combinations.

In a fourth embodiment of the invention, ziprasidone is solubilized using a combination of cyclodextrin and one or more co-solvents in which said ziprasidone is soluble. Without limitation, a mixture of cyclodextrin such as SBECD and a co-solvent or co-solvents, such as a pyrrolidone or mixture of pyrrolidones, for example 2-pyrrolidone and/or NMP, in water, can be used to form the solubilized ziprasidone of the invention. Suitable viscosity agents, such as polyethylene glycol (PEG), can be employed to form the injectable depot formulation of the invention. For example, solutions of up to about 140 mgA/ml ziprasidone mesylate salt can be prepared using 60% NMP/water with 40% SBECD with 10% PEG (e.g. PEG 3350); in another practice of this embodiment, a 140 mgA/ml solution of ziprasidone can be prepared using 60% 2-pyrrolidone/water with 40% SBECD and 30% PEG 3350 as viscosity agent. In an optional practice, a crystallization inhibitor, such as PVP 30, at e.g. up to about 70 mg/ml, can be added. In another aspect of this embodiment of the invention, a non-aqueous depot formulation can be prepared in accordance with the invention by utilizing the co-solvents above with non-aqueous but polar solvents such as benzyl benzoate (BB) and the like. For example, a 140 mgA/ml ziprasidone formulation can be prepared using 30% BB, 70% 2-pyrrolidone with 40% SBECD, the formulation having a viscous gel-like consistency suitable for a depot effect.

Additionally, pH modifiers known in the art to be acidic in nature may be employed in any of the foregoing formulations.

The following examples are illustrative only; they are not to be construed as limiting of the scope or spirit of the invention.

### EXAMPLE 1

This example demonstrates an embodiment of the invention wherein the depot formulation comprises ziprasidone solubilized with cyclodextrin and having a cellulose derivative as a viscosity agent forming an aqueous suspension.

175 mgA of ziprasidone powder in the form of ziprasidone mesylate trihydrate was provided. The ziprasidone powder was admixed with a vehicle constituted as follows:

SBECD at 30% w/v

Sodium carboxymethyl cellulose (NaCMC) at 0.5% w/v

Polysorbate 80 (Tween 80) at 0.02% w/v

Sterile water for injection q.s. at 2.5 ml.

Total fill of the vehicle was 3 ml. The ziprasidone powder was admixed with 2.3 ml of the vehicle to produce a 2.5 ml aqueous suspension at 70 mgA/ml ziprasidone. The resultant admixture was agitated for 1 minute followed by a 15 minute period of waiting to wet the ziprasidone powder whereafter the admixture was again agitated for an additional minute. A 21 gauge syringe was loaded with 2 ml of the final admixture to provide a dose of 140 mg ziprasidone. Viscosity was about 31 to about 80 cps.

The pharmacokinetic (PK) profile of the foregoing aqueous suspension depot formulation obtained from the kit of the invention was investigated in beagle dogs and compared to the following: Comparative Sample (1): an immediate release formulation comprised of solubilized ziprasidone, but no viscosity agent; and Comparative Sample (2): an aqueous suspension comprised of a viscosity agent (SBECD) and unsolubilized ziprasidone. The 2 ml volumes in all cases were injected intramuscularly and plasma levels measured over time. The results were as follows: Comparative Sample (1) showed no depot effect, i.e. the serum concentration of ziprasidone was not quantifiable after 48 hrs; there was no sustained serum concentration. Comparative Sample (2) showed a ziprasidone serum concentration of 4.6±2.4 ng/ml (mean of 12-336 hrs). The present invention on the other hand showed a ziprasidone serum concentration of 12.9±3.7 ng/ml, which represented an increase in depot effect of approximately 280% over that of the next closest sample, Comparative Sample (2).

Four other ziprasidone aqueous suspension depot formulations, two each providing 140 mgA/ml and 210 mgA/ml, but with different concentrations of cyclodextrin, were prepared as set forth in the following Table 1:

TABLE 1
Various combinations of the two vials and dosing instructions
to prepare 140 and 210 mgA/ml aqueous suspensions with
vehicle containing 10 and 20% SBECD.

| Formulation No. | Vial 1: Drug Powder | Vehicle | Dosing Instruction |
|---|---|---|---|
| 1 | Ziprasidone mesylate | 1.5% NaCMC 7LF, | Constitute and dose |
| 140 mgA/ml in | 735 mgA/vial | 10% SBECD, 0.1% | within 15 to 45 |
| vehicle with 10% | | Tween 80 | minutes |
| SBECD | | 4.6 ml | |
| 2 | Ziprasidone mesylate | 0.5% NaCMC 7H3SF, | Constitute and dose |
| 140 mgA/ml in | 735 mgA/vial | 20% SBECD, 0.1% | within 15 to 45 |
| vehicle with 20% | | Tween 80 | minutes |
| SBECD | | 4.6 ml | |
| 3 | Ziprasidone mesylate | 1.5% NaCMC 7LF, | Constitute and dose |
| 210 mgA/ml in | 735 mgA/vial | 10% SBECD, 0.1% | within 15 to 45 |
| vehicle with 10% | | Tween 80 | minutes |
| SBECD | | 2.9 ml | |
| 4 | Ziprasidone mesylate | 0.5% NaCMC 7H3SF, | Constitute and dose |
| 210 mgA/ml in | 735 mgA/vial | 20% SBECD, 0.1% | within 15 to 45 |
| vehicle with 20% | | Tween 80 | minutes |
| SBECD | | 2.9 ml | |

### EXAMPLE 2

This example demonstrates an embodiment of the invention wherein the depot formulation is a non-aqueous suspension comprising a pre-formed ziprasidone/cyclodextrin complex and having a viscosity agent.

An isolated pre-formed complex of ziprasidone mesylate trihyrate and SBECD was prepared as follows:

A 1095.3 gm batch of solution was prepared in an 80° C. water bath. After SBECD was dissolved in sterilized water for injection (SWFI) ziprasidone mesylate trihydrate was added to the resulting solution. During the entire process, the solution was stirred magnetically. The drug solution (82 mgA/ml) was filtered through a 0.45 μm filter and 2 ml aliquots were pipetted into 20 ml vials.

The vials of solution prepared above were lyophilized to obtain the ziprasidone-SBECD complex as a freeze dried solid. A lyophilization cycle was used with the following conditions: 1) Freezing step: temperature was −55° C. at 1° C./minute; 2) Primary drying: from −55° C. to −32° C. at 0.05° C./minute, held at −32° C. for 7 days, vacuum 100 mTorr; 3) Secondary drying: from −32° C. to 8° C. at 0.1° C./minute, held at 8° C. for 20 hours, vacuum 70 mTorr, then from 8° C. to 30° C. at 0.1° C./minute, held at 30° C/ for 20 hours, vacuum 70 mTorr. The complex was comprised of ziprasidone at approximately 80 mgA/ml with about 56% SBECD.

Samples of the lyophilized complex were suspended in the various biocompatible, sustained release non-aqueous vehicles. The mean serum concentration of ziprasidone over a 12-336 hour period achieved in beagle dogs who had received non-aqueous depot suspensions are shown below in Table 2.

TABLE 2

| Formulation | | Mean Serum |
|---|---|---|
| No. | Depot Formulation | Concentration (ng/ml) |
| 1 | Suspension in 2% Aluminum | Depot = 18 ng/ml |
| | Monostearate (ALMS) | |
| | gelled Sesame oil | |
| | (60 mgA/ml; 2 ml injection) | |
| 2 | Suspension in 100-300 mg | Depot = 18.76 ng/ml |
| | Stearic acid (SA) in NMP | |
| | (70 mgA/ml; 2 ml injection) | |

### EXAMPLE 3

This example demonstrates an embodiment of the invention wherein the depot formulation comprises ziprasidone solubilized with cyclodextrin where the cyclodextrin also serves as the viscosity agent. This particular example employs a high concentration of SBECD to form an aqueous solution of ziprasidone at about 80 mgA/ml with about 56% SBECD. To facilitate drug dissolution, a pre-weighed amount of SBECD (gram weight SBECD equivalent to ml of depot formulation being prepared) was dissolved in water by heating in a water bath at 50° C. Ziprasidone mesylate was added in about 50 mg increments while heat was supplied to maintain the system at 50-60° C. A total of 572.99 mg Ziprasidone mesylate was added to 3 ml of 100% SBECD solution resulting in formation of 140 mgA/ml (191 mg/ml) clear viscous solution. The above solution was cooled to room temperature, and the solution remained clear for 2 weeks. Due to volume expansion, the final concentration of ziprasidone was about 80 mgA/ml with 56% cyclodextrin.

Preparation of the above formulation was scaled up as follows to prepare a stock solution and to analyze the volume expansion and measure ziprasidone concentration using HPLC:

The stock solution was prepared using the same method as described above, however due to the higher solution volume (20 ml), the dissolution time was much longer (over 4 hours) even though micronized ziprasidone mesylate was used. During compounding, significant volume expansion was noted. To correct for the volume expansion, the specific gravity of the solution was determined to be 1.188 gm/ml. The volume of water used to prepare this solution was 20 ml, however, the final volume of the solution was 36.6 ml, and the weight of the solution was 43.5 gm. Therefore, taking into consideration 83% volume expansion, the corrected concentrations of drug and SBECD in this solution are 77 mgA/ml and 55% w/v, respectively. HPLC analysis of this solution by the potency method showed a potency of 75 mgA/ml (102.3 mg/ml), and no degradation products were detected.

The above two preparations resulted in ziprasidone solution at 77 mgA/ml with 55% SBECD indicating significantly higher solubility of ziprasidone with relative lower molar ratio of SBECD to drug (1.3:1), more than would be predicted based on a linear phase solubility diagram of ziprasidone and SBECD. The extent of solubilization was further confirmed by preparation of 82 mgA/ml ziprasidone solution with 59% SBECD using the same method. Viscosity was greater than 160 cps.

What is claimed is:

1. An injectable depot formulation comprising:
a solubilized aryl-heterocyclic compound; and a viscosity agent.

2. The injectable depot formulation of claim 1 wherein said aryl-heterocyclic compound is ziprasidone.

3. The injectable depot formulation of claim 2 wherein said aryl-heterocyclic compound is solubilized with a cyclodextrin.

4. The injectable depot formulation of claim 2, optionally comprising a crystallization-inhibitor and wherein said cyclodextrin is present in a concentration of greater than about 50% w/v.

5. The injectable depot formulation of claim 3, optionally comprising a crystallization-inhibitor and wherein said cyclodextrin is present in a concentration of greater than about 50% w/v.

6. The injectable depot formulation of claim 2 wherein said viscosity agent comprises a cellulose derivative, polyvinylpyrrolidone, alginates, chitosan, a dextran, gelatin, polyethylene glycols, polyoxyethylene ethers, polyoxypropylene ethers, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers or terpolymers of the foregoing, sucrose acetate isobutyrate, PLGA, stearic acid/NMP, or a combination thereof.

7. The injectable depot formulation of claim 3 wherein said viscosity agent comprises a cellulose derivative, polyvinylpyrrolidone, alginates, chitosan, a dextran, gelatin, polyethylene glycols, polyoxyethylene ethers, polyoxypropylene ethers, polylactides, polyglycolides, polycaprolactones, polyanhydrides, polyamines, polyurethanes, polyesteramides, polyorthoesters, polydioxanones, polyacetals, polycarbonates, polyorthocarbonates, polyphosphazenes, succinates, polycarbonates, poly(maleic acid), poly(amino acids), polyhydroxycellulose, chitin, copolymers or terpolymers of the foregoing, sucrose acetate isobutyrate, PLGA, stearic acid/NMP, or a combination thereof.

8. The injectable formulation of claim 6 wherein said cellulose derivative includes methyl cellulose, sodium carboxymethyl cellulose or hydroxypropyl methyl cellulose, and said polylactides, polyglycolides, copolymers or terploymers thereof includes poly-lactic-co-glycolic acid.

9. The injectable depot formulation of claim 3 wherein said cyclodextrin is γ-cyclodextrin, β-cyclodextrin, HPBCD, SBECD or a mixture thereof.

10. The injectable depot formulation of claim 3 wherein said solubilized ziprasidone comprises a pre-formed complex with said cyclodextrin.

11. The injectable depot formulation of claim 3 further comprising water; optionally a crystallization inhibitor; and a co-solvent comprising a pyrrolidone or mixture of pyrrolidones.

12. The injectable depot formulation of claim 3 further comprising a non-aqueous polar solvent.

13. The injectable depot formulation of claim 2 wherein said formulation has a viscosity of greater than about 3.2 cps.

14. The injectable depot formulation of claim 3 wherein said formulation has a viscosity of greater than about 3.2 cps.

15. A depot formulation for intramuscular injection comprising ziprasidone mesylate solubilized with SBECD; and a viscosity agent.

16. The depot formulation of claim 15 wherein said SBECD is present at a concentration of from about 5% w/v to about 35% w/v and wherein said viscosity agent is sodium carboxymethyl cellulose in an aqueous vehicle.

17. A depot formulation for intramuscular injection comprising:
ziprasidone mesylate in an amount sufficient to provide at least about 10 mgA to about 30 mgA per day of ziprasidone for at least about 8 hours to about 2 weeks, said ziprasidone mesylate solubilized with SBECD, said SBECD present at a concentration of about 5% to about 35% w/v;
sodium carboxymethyl cellulose present in a concentration of about 0.25% w/v to about 2% w/v;
optionally a pharmaceutically acceptable surfactant present in an amount of up to about 1%; and
water.

18. A method of treating a psychotic disorder, for example schizophrenia comprising:
administering by intramuscular injection to a patient in need of such treatment a depot formulation comprised of ziprasidone in an amount sufficient to provide at least about 10 mgA to about 30 mgA per day of said ziprasidone for at least about 8 hours to about 2 weeks, said ziprasidone solubilized with SBECD, said formulation further comprising a viscosity agent.
